(19) United States
(12) Patent Application Publication
(10) Pub. No.: US 2010/0179318 A1
(43) Pub. Date: Jul. 15, 2010

(54) PROCESS TO MAKE LESTAURTINIB

(75) Inventor: Eric J. Stoner, Kenosha, WI (US)

Correspondence Address: PAUL D. YASGER;ABBOTT LABORATORIES, 100 ABBOTT PARK ROAD, DEPT. 377/AP6A, ABBOTT PARK, IL, 60064-6008, US

(73) Assignee: ABBOTT LABORATORIES, Abbott Park, IL (US)

(21) Appl. No.: 12/522,993

(22) PCT Filed: Jan. 11, 2008
(86) PCT No.: PCT/US08/50830

§ 371 Date: Mar. 10, 2010

Related U.S. Application Data
(63) Continuation-in-part of application No. 11/972,060, filed on Jan. 10, 2008, now abandoned.
(60) Provisional application No. 60/884,550, filed on Jan. 11, 2007.

(51) Int. Cl. C07D 498/22

(52) U.S. Cl. 540/545

(57) ABSTRACT

A process to make lestaurtinib, also known as (9S-(9α,10β,12α))-2,3,9,10,11,12-hexahydro-10-hydroxy-10-(hydroxymethyl)-9-methyl-9,12-epoxy-1H-diindolo[1,2,3-fg:3′,2′,1′-kl]pyrrolo[3,4-i][1,6]benzodiazocin-1-one is disclosed.

### FIELD OF THE INVENTION

This invention pertains to a process to make lestaurtinib, also known as (9S-(9α,10β,12α))-2,3,9,10,11,12-hexahydro-10-hydroxy-10-(hydroxymethyl)-9-methyl-9,12-epoxy-1H-diindolo[1,2,3-fg:3′,2′,1′-kl]pyrrolo[3,4-i][1,6]benzodiazocin-1-one (CAS Registry No. 111358-88-4).

### BACKGROUND OF THE INVENTION

Lestaurtinib is an semi-synthetic, orally bioavailable receptor-tyrosine kinase inhibitor that has been shown to have therapeutic utility in treating diseases such as acute myeloid leukemia, chronic myeloid leukemia and acute lymphocytic leukemia. It is a synthetic derivative of K-252a, a fermentation product of Nonomurea longicatena, and belongs to a class of indolocarbazole alkaloids. A synthesis of lestaurtinib, also known as (9S-(9α,10β,12α))-2,3,9,10,11,12-hexahydro-10-hydroxy-10-(hydroxymethyl)-9-methyl-9,12-epoxy-1H-diindolo[1,2,3-fg:3′,2′,1′-kl]pyrrolo[3,4-i][1,6]benzodiazocin-1-one (CAS Registry No. 111358-88-4), is reported in U.S. Pat. No. 4,923,986. The '986 synthesis uses lithium aluminum hydride at low temperature, reaction conditions that are not easily scalable due to the necessity of lower temperature and reactivity of the reductant. There is therefore an existing need in the chemical process arts for a scalable process for making lestaurtinib that avoids the necessity of lithium aluminum hydride and lower temperatures.

### SUMMARY OF THE INVENTION

One embodiment of this invention, therefore, comprises a process for making lestaurtinib (I)

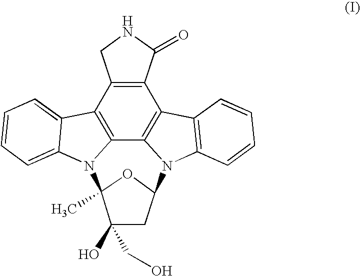

comprising reacting, from about 15° C. to about 45° C., a compound having formula K-252

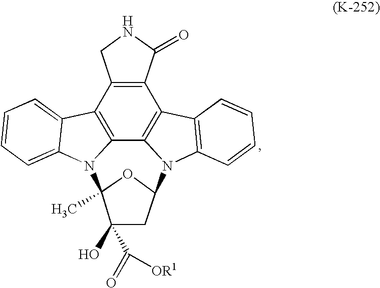

wherein R¹ is alkyl, alkenyl, alkynyl, aryl, heteroaryl, cycloalkyl, cycloalkyl or alkyl, alkenyl or alkynyl, each of which is substituted with aryl, heteroaryl or cycloalkyl
and a borohydride reagent in a solvent comprising an aromatic hydrocarbon and an alcohol having formula R²OH, wherein R² is alkyl; and isolating the lestaurtinib.

Still another embodiment comprises a process for making lestaurtinib comprising reacting, from about 15° C. to about 45° C., a compound having formula K-252a

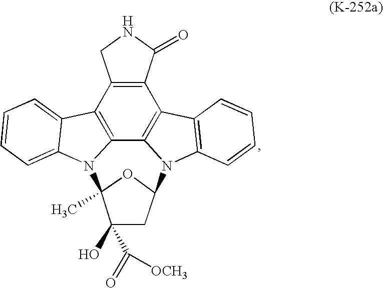

and a borohydride reagent in a solvent comprising toluene and an alcohol having formula R²OH; and isolating the lestaurtinib.

Still another embodiment comprises a process for making lestaurtinib comprising reacting, from about 15° C. to about 45° C., a compound having formula K-252a

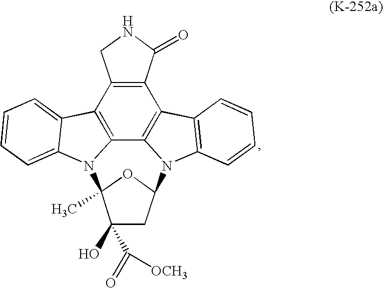

and a borohydride reagent in a solvent comprising toluene and methanol; and isolating the lestaurtinib.

Still another embodiment comprises a process for making lestaurtinib comprising reacting, from about 15° C. to about 45° C., a compound having formula (K-252a)

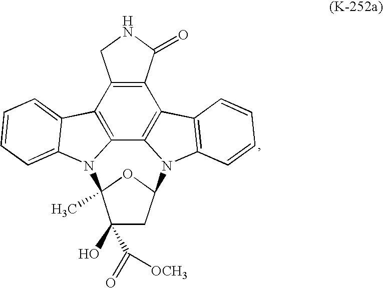

and sodium borohydride in toluene and methanol; and isolating the lestaurtinib.

Still another embodiment comprises any of the foregoing process conducted over about one-half to about ten hours.

Still another embodiment comprises any of the immediately foregoing process conducted over about 6 hours.

### DETAILED DESCRIPTION OF THE INVENTION

This invention pertains to a process to make lestaurtinib comprising reducing an ester-containing intermediate to an alcohol at ambient temperature using a borohydride reagent in a solvent comprising two liquids. U.S. Pat. No. 4,923,986 reports a synthesis of lestaurtinib using an aluminum hydride reagent at low temperature, a less easily scalable process due to the necessity of temperature adjustment, presumably to minimize reduction of a synthetically vulnerable amide moiety also present on the molecule. The process of this invention uses reaction conditions for reducing esters that, taken as a whole, are wholly surprising in view of the '986 patent.

Variable moieties are represented by identifiers (capital letters with numerical and/or alphabetical superscripts) and may be specifically embodied.

It is also meant to be understood that a specific embodiment of a variable moiety may be the same or different as another specific embodiment having the same identifier.

The term “alkenyl,” as used herein, means C₃-alkyl, C₄-alkyl, C₅-alkyl and C₆-alkyl.

The term “alkyl,” as used herein, means C₁-alkyl, C₂-alkyl, C₃-alkyl, C₄-alkyl, C₅-alkyl and C₆-alkyl.

The term “alkynyl,” as used herein, means C₃-alkyl, C₄-alkyl, C₅-alkyl and C₆-alkyl.

The term “aromatic hydrocarbon,” as used herein, means benzene, toluene, ortho-xylene, meta-xylene, para-xylene and 1,3,5-mesitylene.

The term “aryl,” as used herein, means phenyl which is unfused or fused with benzene.

The term “borohydride reagent,” as used herein, means lithium borohydride, sodium borohydride, potassium borohydride and compounds having formula M¹BH₃(OR²), M¹BH₂(OR²)₂ and M¹BH(OR²)₃, wherein M¹ is lithium, sodium or potassium.

The term “cycloalkyl,” as used herein, means C₃-cycloalkyl, C₄-cycloalkyl, C₅-cycloalkyl and C₆-cycloalkyl.

The term “heteroaryl,” as used herein, means furanyl, imidazolyl, isothiazolyl, isoxazolyl, 1,2,3-oxadiazoyl, 1,2,5-oxadiazolyl, oxazolyl, pyrazinyl, pyrazolyl, pyridazinyl, pyridinyl, pyrimidinyl, pyrrolyl, tetrazolyl, thiazolyl, thiophenyl, triazinyl and 1,2,3-triazolyl.

It is meant to be understood that, in a preferred embodiment for the practice of this invention, the borohydride reagent formed by addition of lithium borohydride, sodium borohydride, potassium borohydride or calcium borohydride to an alcohol may be a combination comprising M¹BH₃(OR²), M¹BH₂(OR²)₂ and M¹BH(OR²)₃.

It is also meant to be understood that the aromatic hydrocarbon and an alcohol having formula R²OH of this invention may have limited solubility in one another, however, the aformentioned solubility will not preclude separation of the two components.

The term “isolating the lestaurtinib,” as used herein, means separating lestaurtinib from impurities. Separating lestaurtinib from impurities is typically accomplished by means such as centrifugation, filtration with or without vacuum, filtration under positive pressure, distillation, evaporation, crystallization, constant volume distillation, extraction, filtration through acidic, basic or neutral alumina, filtration through acidic, basic or neutral charcoal, filtration through a porous paper, plastic or glass barrier, column chromatography on silica gel, ion exchange chromatography, recrystallization, normal-phase high performance liquid chromatography, reverse-phase high performance liquid chromatography, trituration, combinations thereof and the like.

The following example is meant to further embody the compounds and processes of this invention.

### EXAMPLE 1

K-252a was treated with a mixture of methanol (2 Kg/Kg K-252a) and toluene (8 Kg/Kg K-252a) at ambient temperature to produce a slurry to which was added sodium borohydride caplets (0.16 Kg/Kg K-252a) in portions over 6 hours. The solids in the reactor dissolved to provide a biphasic solution which was stirred for 3 hours, after which time 0.2% of K-252a remained. The lower layer was isolated, diluted with methanol (11 Kg/KG K-252a), and quenched with glacial acetic acid (0.41 Kg/Kg K-252a), which caused a small amount of amorphous lestaurinib to form. Stirring at room temperature converted the amorphate to lestaurinib methanolate. The methanol solvate was collected by vacuum filtration and washed with methanol (2 Kg/Kg K-252a) and water (2 Kg/Kg K-252a). One third of the methanol solvate was dissolved in acetone (36 Kg/Kg K-252a) and methanol (24 Kg/Kg K-252a) at 55° C. The solution was polish filtered through a series of polypropylene filters. Following filtration it was diluted with isopropanol (12 Kg/Kg K-252a) and distilled to one-half volume at atmospheric pressure. One-half of the remaining methanol solvate was dissolved in acetone (36 Kg/Kg K-252a) and methanol (24 Kg/Kg K-252a) at 55° C. This solution was polish filtered and added to the first distillation solution. Following dilution with isopropanol (12 Kg/Kg K-252a), the combination was distilled to half volume at atmospheric pressure. The remaining methanol solvate was dissolved in a mixture to acetone (36 Kg/Kg K-252a) and methanol (24 Kg/Kg K-252a) at 55° C. This solution was also polish filtered and added to the distillation residue. Following dilution with isopropanol (12 Kg/Kg K-252a), the combination was distilled to half volume at atmospheric pressure. During distillation, solids precipitated from the solution. The distillation was continued at constant volume with isopropanol addition until an internal temperature of 82° C. was achieved for one hour. After cooling to 0° C., the mixture was filtered. The filtrant was washed with isopropanol (2 Kg/Kg K-252a), and the solids were dried under vacuum at 75° C.

The foregoing is meant to be illustrative of this invention and not limiting. Obvious variations and changes are meant to be within the scope of this invention, as defined in the claims.

We claim:

1. A process for making lestaurtinib (I) 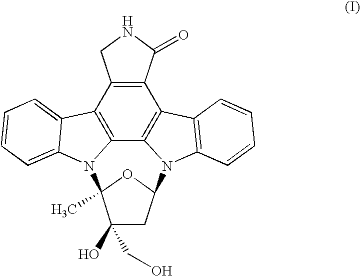
comprising reacting, from about 15° C. to about 45° C., a compound having formula K-252 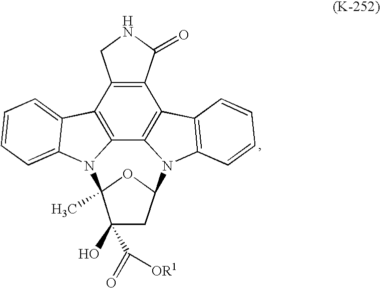
wherein R¹ is alkyl, alkenyl, alkynyl, aryl, heteroaryl, cycloalkyl, cycloalkyl or alkyl, alkenyl or alkynyl, each of which is substituted with aryl, heteroaryl or cycloalkyl,
and a borohydride reagent in a solvent comprising an aromatic hydrocarbon and an alcohol having formula R²OH; and isolating the lestaurtinib.

2. The process of claim 1 conducted over about one-half to about ten hours.

3. The process of claim 2 conducted over about six hours.
